(19) United States
(12) Patent Application Publication
(10) Pub. No.: US 2003/0144641 A1
(43) Pub. Date: Jul. 31, 2003

(54) Absorbent products having high total front pad AULs

(76) Inventors: Dennis Chia-Bin Chen, Alpharetta, GA (US); Andrew Baker, Lawrenceville, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 10/058,065

(22) Filed: Jan. 29, 2002

(51) Int. Cl. A61F 13/15

(52) U.S. Cl. 604/368; 604/378

(57) ABSTRACT

An absorbent core for use in an absorbent article is disclosed which has a front pad comprising a two inch diameter circle having substantially as its center the insult point that is capable of absorbing 32 grams or more of an aqueous solution comprising 1.0 weight % sodium chloride after ten minutes of contact with the solution while under a constant restraining pressure of about 0.5 psi. The absorbent core provides improved protection against leakage in an absorbent article containing the core. A method of designing, optimizing, and predicting good leakage protection in an absorbent article is also disclosed.

### BACKGROUND OF THE INVENTION

1. Field of the Invention

The invention relates generally to the field of absorbent cores for assembly into absorbent articles, and specifically to the field of absorbent cores with high front pad total absorptive capacity for use in the manufacture of absorbent articles. Absorbent articles having absorbent cores with high front pad total absorptive capacity exhibit improved protection for leakage with the same or lower total amount of expensive superabsorbent material in the core when compared to more leakage-prone absorbent articles. A method for reducing leakage from an absorbent article by including therein an absorbent core with high front pad total absorptive capacity also is disclosed, as well as a method for predicting the leakage performance, and a method for designing an absorbent article based on core properties.

2. Description of Related Art

Disposable absorbent garments such as infant diapers or training pants, adult incontinence products, and other products are well-known in the art for the containment of body exudates. It is estimated that approximately 65% of absorbent articles are used for the containment of urine only. Typically, the chassis of such absorbent garments comprises a liquid-permeable body-contacting liner sheet (or “top sheet”), a liquid-impermeable backing sheet (or “back sheet”), and a moisture-absorbent core (or “absorbent core”). The absorbent core usually is made of a nonwoven composite of randomly arrayed fiber and superabsorbent polymer material (“SAP” or “superabsorbent material”) with optional additional fibrous and/or particulate fillers to enhance SAP performance, and is generally disposed between the top sheet and the back sheet.

Disposable absorbent garments typically are prepared by continuously supplying the various components of the garment, and assembling these components into the final garment. Methods of bonding the different parts of the absorbent garment to form a finished garment are well known in the art. Various attachment mechanisms used for bonding different parts of the absorbent garment to form the finished garment include using a uniform continuous layer of adhesive, a patterned layer of adhesive, or an array of separate lines, spirals, or spots of adhesive. Alternatively, the attachment mechanism may comprise heat bonds, pressure bonds, chemical or solvent boding, ultrasonic bonding or welding, stitching, dynamic mechanical bonds, autogenous bonding, or any other suitable attachment mechanism or combinations of these attachment mechanisms as are known in the art.

The absorbent core typically is formed from a core composite material, which may be purchased as bulk roll goods. Alternatively, the core composite material may be formed in a forming chamber from a fiberized material (or “fluff”) with incorporated SAP particles, which then may be encased in a liquid pervious wrap to form and stabilize the layer or layers of the absorbent core.

The use of SAP particles, which can absorb many times their own dry weight in liquid, in core composite materials to improve liquid absorption and protection against leakage is well known in the art, and has allowed the manufacture of absorbent articles that are significantly less bulky and yet display equal and even improved performance to older, more bulky styles of such articles. Various absorbent composites for use in making absorbent cores are disclosed in U.S. Pat. No. 4,076,663, issued to Masuda, et. al.; U.S. Pat. No. 4,286,082, issued to Tsubakimoto, et. al.; U.S. Pat. No. 4,062,817, issued to Westerman; U.S. Pat. No. 4,340,706, issued to Obayashi, et. al.; and U.S. Pat. No. 4,535,098, issued to Evani et. al. The disclosures of each of these patents are hereby incorporated by reference in their entireties and in a manner consistent with this application. For example, Masuda et. al. discloses a biodegradable water absorbent resin made by polymerizing at least one polysaccharide, at least one monomer having a polymerizable double bond which is water soluble or becomes water soluble by hydrolysis, and a cross-linking agent. Tsubakimoto et. al. discloses an absorbent resin composite obtained by copolymerizing a mixture of an acrylate salt monomer with a crosslinkable monomer in the presence of at least one surface-active agent. Westerman et. al. discloses a polymer of unsaturated copolymerizable carboxylic acids, at least one acrylic or methacrylic ester, and another acrylic or methacrylic ester, optionally with a cross-linking agent. Obayashi et. al. discloses an alkali metal acrylate polymer cross-linked with a cross-linking agent. Evani et. al. discloses a polymerized water-soluble monomer and water-insoluble monomer having a pendant hydrophobic moiety, with an optional cross-linking monomer.

The use of SAP particles in core composite materials has increased the absorptive capacity of absorbent cores made using these core composite materials for liquid body exudates, as urine. However, at times SAP particles are unable to absorb liquid as rapidly as the liquid is applied, particularly when electrolytes are present in the liquid (as in urine). Also, the phenomenon of gel blocking can occur when SAP particles swell and block the interstitial spaces of the fibers that form the rest of the core composite material, preventing excess liquid from spreading to other portions of the absorbent core to be absorbed.

Although the use of higher amounts of SAP particles in core composite materials would seem to improve the absorptive capacity for liquid body exudates of an absorbent core made from these core composite materials, SAP usage in the absorbent core is a significant cost to the production of the absorbent article and thus must be optimized to provide cost-effective leakage protection through efficient placement of the SAP. Several approaches have been examined to improve efficient use of SAP, such as the use of core structures designed to more quickly distribute liquid and the use of high absorbent superabsorbent materials with particular particle size distributions to more quickly absorb liquid.

Absorbent structures and absorbent garments that exhibit improved fluid absorption capacities by improved wicking and distribution of fluids are disclosed in U.S. Pat. No. 3,901,236, issued to Assarsson et. al.; U.S. Pat. No. 4,834,735, issued to Alemany et. al.; U.S. Pat. No. 4,699,619, issued to Bernardin; and U.S. Pat. No. 4,798,603, issued to Meyer et. al., the disclosures of which are hereby incorporated by reference in their entireties and in a manner consistent with this application. Assarsson discloses the use of hydrogel composites having improved fluid absorption capacities in disposable absorbent articles. Bernardin discloses an absorbent structure for use in absorbent pads for incorporation in absorbent garments, where the absorbent structure may have a superabsorbent material that wicks fluid from the surface and transfers it to another element of the structure. Alemany discloses an absorbent garment with an absorbent member that comprises an acquisition zone containing absorbent gelling material that can rapidly acquire discharged liquids and distribute those liquids within the absorbent member.

Faster-absorbing core composite materials are disclosed in U.S. Pat. Nos. 5,147,343 and 5,149,335, both of which were issued to Kellenberger, et. al. (collectively “Kellenberger”), the disclosures of which are hereby incorporated by reference in their entireties and in a manner consistent with this disclosure. By incorporating superabsorbent materials that can quickly absorb a high level of liquid into the absorbent core, where the particle size distribution of the superabsorbent particles have a certain relationship to the pore sizes of the matrix of the composite, the core composite materials described in these disclosures exhibit an improved ability to swell against a constant applied pressure. The ability of a superabsorbent material to swell against an applied pressure is measured by standard Absorbancy Under Load (“AUL”) tests well known to those skilled in the art.

Other improvements are disclosed in U.S. Pat. No. 5,601,542, issued to Melius, et. al. (“Melius”), the disclosure of which is hereby incorporated by reference in its entirety and in a manner consistent with this disclosure. Melius describes the use of superabsorbent materials having a certain Pressure Absorbency Index (“PAI”), where PAI is defined as the sum of AUL values measured at a range of applied pressures. Kellenberger and Melius describe the incorporation of superabsorbent materials in core composite materials that can rapidly absorb liquids while under a restraining pressure, i.e., that absorb high levels of liquid during the first sixty (60) minutes of exposure to the liquid.

### SUMMARY OF THE INVENTION

It would be desirable to provide an absorbent article that provides improved protection against urine leakage without increasing the amount of SAP used in the absorbent core of the article, or without using more costly, faster-absorbing SAP in the absorbent core. It also would be desirable to provide a method to produce such an absorbent article, as well as a method to predict the leakage performance and to aid in the design of an absorbent core based on the total absorptive capacity of the front pad of the core.

The inventors have found that superabsorbent materials may be used in core composite materials of absorbent cores to provide improved urine leakage protection while maintaining the cost-effectiveness of the absorbent article. The superabsorbent materials of the invention have been found to be most effectively used in the core composite materials when the front pad of the absorbent article, substantially in or around the insult point, has a high total absorptive capacity that may be measured by modified AUL testing as is known in the art. An average total absorptive capacity for the front pad of the absorbent article of about 32 grams or more of a 1.0 weight % NaCl solution absorbed after ten (10) minutes of contact with the solution while under a restraining pressure of about 0.5 psi provides improved protection against urine leakage for the absorbent article, virtually regardless of the SAP concentration or absorptive capacity of the rest of the article. By effectively designing the total absorptive capacity of the absorbent article in the front pad, overall SAP usage in the core may be kept to a cost-effective level while improving the performance of the absorbent article against leakage.

High front pad total absorptive capacity may be achieved using numerous techniques, such as, for example, using high AUL SAP particles in the core composite material, or by using high weight ratios of SAP particles to fluff, in the front pad of the absorbent core. The total amount of SAP can be maintained at the same amount as used in lesser-performing absorbent articles by distributing the SAP within the core composite material of the absorbent core to achieve these values. The invention therefore achieves an efficient use of SAP.

It therefore is a feature of the invention to provide an absorbent article with improved protection from leakage by incorporating therein an absorbent core having high front pad total absorptive capacity, as measured by absorptive capacity testing of the absorbent article. It is another feature of the invention to provide an absorbent core for use in an absorbent article that cost-effectively uses SAP particles to provide improved protection against urine leakage. It is yet another feature of the invention to provide improved protection against leakage without increasing the amount of SAP in an absorbent core. It is yet another feature of the invention to provide a method of designing, predicting, and achieving optimum leakage performance of an absorbent article based on the absorptive capacity within the front pad of the article.

In accordance with these and other features of the various embodiments of the invention, there is provided an absorbent article including a front pad having a high total absorptive capacity that provides improved protection against leakage. High front pad total absorptive capacity may be achieved by using high AUL SAP particles, or by using high weight ratios of SAP particles-to-fluff, in the front pad of the absorbent core. Front pad total absorptive capacity is the capacity for liquid absorption in and around the insult point of the absorbent article and is determined by measuring the absorptive capacity of the front pad of the article by slightly modifying presently known AUL testing techniques. The desired total absorptive capacity of the front pad of the absorbent core should be, on average, about 32 grams or more of a 1.0 weight % NaCl aqueous solution absorbed after ten (10) minutes of contact with the solution under a constant restraining pressure of about 35,000 dynes/cm², or about 0.5 lbs/in². The average total absorptive capacity of the front pad may be determined by measuring the absorptive capacity of one or more samples taken from substantially within a two (2) inch diameter circle having as its center the insult point of the absorbent article.

In accordance with another feature of the invention, there is provided an absorbent core that includes SAP particles having a high AUL value in the front pad that exhibits improved protection from leakage, with the front pad total absorptive capacity about 32 grams or more of a 1.0 weight % NaCl solution after ten (10) minutes of contact with the solution under a restraining pressure of about 0.5 psi.

In accordance with yet another feature of the invention, there is provided an absorbent core that includes a weight ratio of SAP to fluff in the front pad that exhibits improved protection from leakage, with the front pad total absorptive capacity about 32 grams or more of a 1.0 weight % NaCl solution after ten (10) minutes of contact with the solution under a restraining pressure of about 0.5 psi.

In accordance with yet another feature of the invention, there is provided an absorbent core that includes a differential distribution of SAP within the core such that the core provides improved protection against leakage when compared to previously known absorbent cores that contain the same total amount of SAP by weight.

In accordance with an additional feature of the invention, there is provided a method for designing, predicting, and achieving optimum leakage performance of an absorbent core by testing the total absorptive capacity substantially in and around the front pad of an absorbent article. Finally, in accordance with yet an additional feature of the invention, a method is provided that includes assembling at least a top sheet material, a back sheet material, and an absorbent core that provides improved leakage protection. The absorbent core used in this embodiment of the invention preferably has a high total absorptive capacity substantially in or around the front pad, as measured by testing the front pad within a two (2) inch diameter circular having substantially as its center sample the insult point of the article, using a modification of known AUL testing techniques.

These and other features and advantages of the invention will be apparent to one skilled in the art upon reading the detailed description that follows.

### BRIEF DESCRIPTION OF THE DRAWINGS

The invention will be described with reference to the accompanying drawings, in which like elements are depicted using like numerals.

FIG. 1 is a schematic of one embodiment of the invention, shown with the elastic elements in their extended condition for clarity.

FIG. 2 is a cut-away side view of the embodiment of FIG. 1 along line A-A.

FIG. 3 is a diagram of a diaper illustrating the insult point.

FIG. 4 is an illustration of a sample holder and sample box useful in carrying out the test method to determine the total absorptive capacity of the front pad of an absorbent article.

FIG. 5 is a diagram of a diaper illustrating an alternative pattern for cutting samples to measure front pad total absorptive capacity.

FIG. 6 is a graph depicting the relationship between front pad total absorptive capacity and percent urine leakage.

### DETAILED DESCRIPTION OF THE PREFERRED EMBODIMENTS

“Absorbent article,” or “absorbent garment,” as used herein, refers to articles and garments that absorb and contain body exudates, and more specifically refers to articles and/or garments that are placed against or in proximity to the body of the wearer to absorb and contain the various exudates discharged from the user's body. A non-exhaustive list of examples of “absorbent articles” includes diapers, diaper covers, disposable diapers, training pants, feminine hygiene products, and adult incontinence products. The invention can be used with all of the foregoing classes of absorbent articles and garments, without limitation, whether disposable or otherwise. Furthermore, the invention will be understood to encompass, without limitation, all classes and types of absorbent articles and garments, including those described above.

The term “SAP” denotes superabsorbent materials that exhibit an ability to absorb many times their own dry weight in liquid. Preferably, the superabsorbent material is a water-insoluble but water-swellable polymeric substance capable of absorbing liquid in an amount which is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers crosslinked with a polyfunctional reactant. Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the tradename SANWET® from Hoechst Celanese Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; FAVOR manufactured by Stockhausen Inc.; HYSORB, available from BASF Aktiengesellschaft, Ludwigshafen, Germany; AQUA KEEP SA60S, manufactured by Seitetsu Kagaku Co., Ltd.; DIAWET, commercially available from Mitsubishi Chemicals, Japan; FLOSORB, available from SNF Floerger, France, AQUALIC, available from Nippon Shokubai, Osaka, Japan.

The term “AUL” denotes Absorbancy Under Load, a standard test known to those skilled in the art that measures the ability of a superabsorbent material to absorb liquid while under a restraining pressure after one hour of contact with the liquid. Methods of measuring AUL at various loads are described in detail in U.S. Pat. Nos. 5,147,343 and 5,149,335, the disclosures of both of which are hereby incorporated by reference in their entireties and in a manner consistent with this disclosure.

The term “total absorptive capacity” denotes the total absorptive capacity of an absorbent article or areas of an absorbent article, measured in terms of grams of a 1.0 weight % NaCl solution absorbed. Total absorptive capacity is measured after about ten (10) minutes of contact with the solution while under a restraining pressure of about 0.5 psi (about 35,000 dynes per cm²).

The term “core composite material” denotes the composite of materials assembled to form the absorbent layer of the absorbent core. Core composite material may comprise tow fibers or other fibrous material (“fluff”), SAP, and other additives such as cellulose acetate fibers, rayon fibers, Courtauld's LYOCELL™ fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof to maintain high SAP efficiencies and provide desired core properties. Particulate additives may also be added to the core composite material in addition to or as a substitute for the foregoing fibrous additives. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity, hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. Fibrous and particulate additives can be used alone or together in the absorbent composites of the invention. Examples of SAP/particulate and SAP/fiber/particulate additives include those described in, for example, U.S. Pat. No. 6,068,620, the disclosure of which is incorporated by reference herein in its entirety, and in a manner consistent with this disclosure.

The term “front pad” denotes an area of an absorbent article comprising a two inch diameter circle having substantially as its center the insult point. The term “insult point” denotes the probable point of an absorbent article proximate to a wearer's body where a majority of the body exudate, and particularly urine, encounters the absorbent core after being discharged from the body of the wearer of the absorbent article. High total absorptive capacity is desired in and around the insult point to reduce exposure of the wearer's skin to body exudate while the exudate is being absorbed into and distributed throughout the absorbent core. The insult point may be found in different regions of the absorbent article depending on the use of the absorbent article. For example, the insult point of absorbent articles used in diapers that are designed to be worn by female babies may differ physically in location from the insult point of absorbent articles used in diapers that are designed to be worn by male babies. Using the guidelines provided herein, those skilled in the art can determine the probable insult point of an absorbent article without undue experimentation.

The expressions “upper layer,” “lower layer,” “above” and “below,” which refer to the various components included in the absorbent core and other components of the invention (including the layers surrounding the absorbent core, if present) are used merely to describe the spatial relationship between the respective components. The upper layer or component “above” the other component need not always remain vertically above the core or component, and the lower layer or component “below” the other component need not always remain vertically below the core or component.

The term “component” can refer, but is not limited, to designated selected regions, such as edges, corners, sides or the like; structural members, such as elastic strips, absorbent pads, stretchable layers or panels, layers of material, or the like; or a graphic.

The term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

The terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Although the various embodiments of the invention are described in the context of a diaper, it is readily apparent and understood that this is not intended to limit the invention.

The invention relates to an absorbent core designed to provide improved leakage protection of body exudates from an absorbent article, particularly urine, by providing a high total absorptive capacity front pad. The desired total absorptive capacity of the front pad may be achieved using several design techniques, including the use of high AUL SAP or a high weight ratio of SAP to fluff in the front pad of the absorbent core. High front pad total absorptive capacity absorbent articles may be achieved by a selective redistribution of the SAP within the absorbent core as compared to lesser-performing absorbent articles. Thus, a higher performing absorbent article with improved leakage protection may be achieved by strategic placement of the SAP to achieve a high absorptive capacity front pad. These results may be achieved without increasing the amount of expensive SAP used to assemble the core, or without using higher absorption and thus more expensive types of SAP in the core.

The invention also relates to a method for predicting the amount of leakage protection that can be achieved by an absorbent core by measuring the total absorptive capacity of the front pad of the absorbent article. The invention also relates to a method of designing and achieving optimum leakage protection in an absorbent article by providing the article with a high total absorptive capacity front pad. Cost efficiency can thus be optimized for the manufacture of absorbent articles while maintaining and improving leakage performance in the absorbent article.

An absorbent article prepared according to one embodiment of the invention includes an absorbent core wherein the front pad total absorptive capacity is about 32 grams or more of a 1.0 weight % NaCl solution after ten (10) minutes of contact with the solution while under a restraining load of about 0.5 psi. The average total absorptive capacity of the front pad may be determined in a preferred embodiment by testing the front pad of the article within a circle two (2) inches in diameter having substantially as its center the insult point, measuring the total absorptive capacity of the sample, and averaging the total absorptive capacities of the samples if more than one sample per article is tested. The total absorptive capacity is then reported for the two (2) inch diameter circular front pad. It is preferred in the invention that the average total absorptive capacity of the sample be equivalent to at least about 32 grams of a 1.0 weight % NaCl solution after ten (10) minutes of contact with the solution while under a restraining pressure of about 0.5 psi.

It has been found that an average front pad total absorptive capacity of about 32 grams or more, preferably about 32 grams to about 70 grams, more preferably about 33 grams to about 60 grams, even more preferably about 34 grams to about 50 grams, and most preferably about 35 grams to about 45 grams, of a 1.0 weight % NaCl solution measured after ten (10) minutes of contact with the solution while under a restraining pressure of about 0.5 psi provides improved protection against leakage for an absorbent article. The front pad is located within a two (2) inch diameter circle, the circle having substantially as its center the insult point. The location of the insult point may differ according to the application of the absorbent article (e.g., intended users may be female or male, or may be a newborn or a walking baby).

In accordance with one embodiment of the invention, the core composite material in the front pad of an absorbent core may comprise a sufficient amount of high AUL SAP particles to achieve the desired front pad total absorptive capacity of about 32 grams or more measured after one hour of contact with a 1.0 weight % NaCl solution while under a restraining pressure of about 0.5 psi. The rest of the core may be designed to be cost-effective; i.e., the rest of the core composite material not including the front pad may comprise lower AUL SAP, or lower amounts of SAP, to minimize the cost of the core.

In one embodiment of the invention, the core composite material in the front pad may contain a high weight ratio of SAP particles to fluff and other fibrous and particulate fillers to achieve the desired absorptive capacity of about 32 grams or more as measured according to the guidelines provided herein. In another embodiment, the ratio of the weight of the core composite material in different areas of the garment may differ such that the front pad has two (2) to four (4) times the amount of core composite material as other regions of the absorbent core.

Substantially the only requirement regarding the SAP itself is that it be sufficient to provide a front pad total absorptive capacity as set forth according to the guidelines in this disclosure.

In another embodiment of the invention, an absorbent article may be assembled using a top sheet made from a top sheet material, a back sheet made from a back sheet material, and an absorbent core disposed between the top sheet and the back sheet, where the absorbent article has a front pad with an absorptive capacity of about 32 grams or more as measured by the guidelines provided herein.

In yet other embodiments of the invention, there are provided a method for designing an absorbent article, a method of predicting the performance of the leakage protection afforded by an absorbent article, and a method of achieving optimum leakage protection in an absorbent article. According to these embodiments, one or more samples of the absorbent article taken from the front pad within a two (2) inch circle substantially centered around the insult point, are tested for total absorptive capacity. The total absorptive capacity of the front pad may comprise the average total absorptive capacity of one or more samples as reported on the basis of the entire front pad. One skilled in the art can design, achieve optimum performance, and/or predict the protection against leakage provided by an absorbent article design within the desired parameters for the particular absorbent article. For example, if cost is the desired design parameter (e.g., provide best performance at lowest cost), an absorbent article can be designed and tested that provides good leakage protection while maintaining a desired cost for assembling the core and absorbent article. Alternatively, an absorbent article may be designed that provides optimum leakage protection using a particular SAP or core composite material by varying the amounts to be used in the front pad to achieve a total absorptive capacity of 32 grams or more as measured according to the guidelines provided herein.

Preferably, the total absorptive capacity of the front pad for an absorbent article designed according to the invention is about 32 grams or more of a 1.0 weight % NaCl solution absorbed, measured after ten (10) minutes of contact with the solution while under a restraining pressure of about 0.5 psi. Although the invention is not to be limited as such, it is believed that testing at about 0.5 psi restraining pressure provides the best conditions for predicting leakage performance under actual use. However, testing may be conducted at other conditions such as 0.3 psi or 0.7 psi so long as the total absorptive capacities would also equate to a value of 32 grams or more as measured after ten (10) minutes of contact with the solution while under a restraining pressure of about 0.5 psi. Those skilled in the art are capable of providing a correlation of total absorptive capacities depending on the restraining pressure, using the guidelines provided herein.

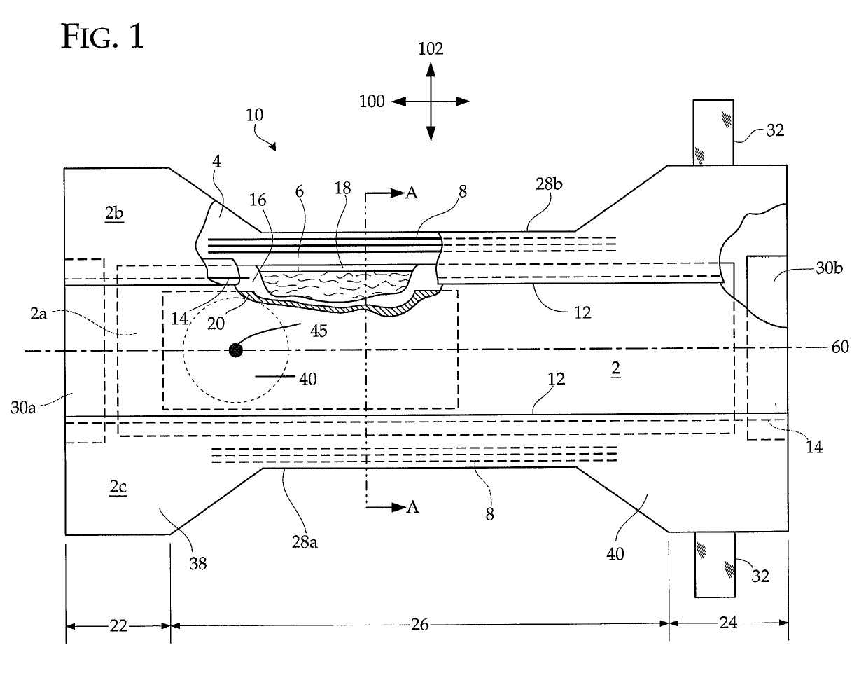

Turning now to the drawings, a preferred embodiment of the invention comprises a disposable absorbent article 10 of the diaper type, such as shown, for example, in FIG. 1. It should be understood, however, that the invention is applicable to other types of absorbent articles. With reference to FIG. 1, the diaper 10 according to a first preferred embodiment is shown with the effects of the elastics removed for purposes of clarity in the description. The diaper 10 has a generally hourglass shape and can generally be defined in terms of a front waist region 22, a back waist region 24, and a crotch region 26. Those skilled in the art will recognize that “front” and “back” are relative terms, and these regions may be transposed without departing from the scope of the invention. Alternatively, the diaper can be configured in a generally rectangular shape or in a “T” shape. A pair of leg openings 28a, 28b extend along at least a portion of the crotch region 26. The diaper preferably comprises a top sheet 2, a back sheet 4, which may be substantially co-terminus with the top sheet 2, and an absorbent core 6 disposed between at least a portion of the top sheet 2 and back sheet 4. One or more pairs of leg elastics 8 (three pairs are shown in FIG. 1) extend adjacent to leg openings 28a, 28b, respectively.

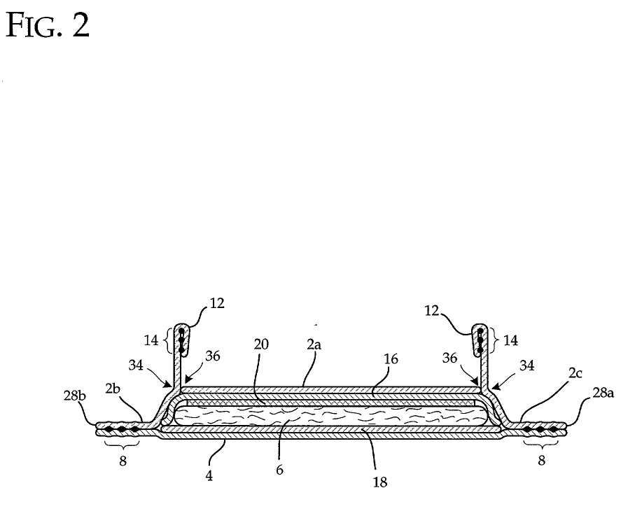

The diaper may further include a front waist elastic system 30a, a back waist elastic system 30b, a fastening system 32 (e.g., tape or other suitable mechanical fastener) and a waste containment system in the form of waste containment flaps 12 (also known as standing leg gathers). Waste containment flaps 12 (FIG. 2) preferably extend from the front waist region 22 to the back waist region 24 along opposite sides of a longitudinal center line or axial center line 60 of the diaper 10, or alternatively only along a portion thereof. The front waist region 22 and rear waist region 24 may include ear portions 38, 40 extending outwardly from the leg openings 28a, 28b.

A variety of back sheet and top sheet constructions and materials are available and known in the art, and the invention is not intended to be limited to any specific materials or constructions of these components. The back sheet 4 may be comprised of any suitable pliable liquid-impervious material known in the art. Typical back sheet materials include films of polyethylene, polypropylene, polyester, nylon, polylactic acid and polyvinyl chloride and blends of these materials. For example, the back sheet can be of a pigmented polyethylene film having a thickness in the range of 0.01-0.03 mm. The moisture-pervious top sheet 2 can be of any suitable relatively liquid-pervious material known in the art that permits passage of liquid therethrough. Non-woven top sheet materials are exemplary because such materials readily allow the passage of liquids to the underlying absorbent core 6. Examples of suitable top sheet materials include non-woven spunbond or carded webs of polypropylene, polyethylene, nylon, polyester and blends of these materials.

The back sheet 4 and the top sheet 2 are “associated” with one another. The term “associated” encompasses configurations whereby the top sheet 2 is directly joined to the back sheet 4 by affixing the top sheet 2 directly to the back sheet 4, and configurations whereby the top sheet 2 is indirectly joined to the back sheet 4 by affixing the top sheet 2 through intermediate members which in turn are affixed to the back sheet 4. While the back sheet 4 and top sheet 2 in the preferred embodiment have substantially the same dimensions, they may also have different dimensions.

The parts may be operatively associated with one another by a variety of methods known in the art, including, but not limited to: using adhesives such as hot melt adhesives and construction adhesives; chemical or solvent bonding; ultrasonic welding; stitching; heat bonding; autogenous bonding; or any other method of affixation known or hereafter discovered. U.S. Pat. No. 4,919,738 issued to Ball et. al. discloses a method of autogenous bonding, and its disclosure is herein incorporated by reference in its entirety in a manner that is consistent with the invention.

The back sheet 4 may be covered with a fibrous, nonwoven fabric such as is disclosed for example in U.S. Pat. No. 4,646,362, which is hereby incorporated by reference and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet may comprise multiple panels, such as three panels wherein a central poly back sheet panel is positioned adjacent the absorbent core while outboard non-woven breathable side back sheet panels are attached to the side edges of the central poly back sheet panel. The back sheet may also be formed from microporous poly coverstock for added breathability. In other embodiments, the back sheet may be a laminate of several sheets. The back sheet may further be treated to render it hydrophilic or hydrophobic, and may have one or more visual indicators associated with it, such as labels indicating the front or back of the diaper or other characters or colorations.

The top sheet 2 may be formed from one or more panels of material and may comprise a laminated sheet construction. In the embodiment of FIG. 1, the top sheet comprises three separate portions or panels. A three-panel top sheet may comprise a central top sheet panel formed from preferably a liquid-pervious material that is either ! hydrophobic or hydrophilic. The central top sheet panel 2a may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel 2a is a cover stock of non-woven material which may be made of carded fibers, either adhesively or thermally bonded, perforated plastic film, spunbonded fibers, or water entangled fibers, which generally weigh from about 10-30 gm/cm² and have appropriate and effective machine direction and cross-machine direction strength suitable for use as a baby diaper cover stock material, as are known in the art. The central top sheet 2a panel preferably extends from substantially the front waist region 22 to the back waist region 24 or a portion thereof.

The second and third top sheet panels, 2b, 2c in this embodiment, may be positioned laterally outside of the central top sheet panel 2a. The outer top sheet panels 2b, 2c are preferably substantially liquid-impervious and hydrophobic, preferably at least in the crotch area. The outer edges of the outer top sheet panels may substantially follow the corresponding outer perimeter of the back sheet 4. The material for the outer top sheet portions or panels is preferably polypropylene and can be woven, non-woven, spunbonded, carded or the like, depending on the application.

Inner regions 34 (FIG. 2) of the outer top sheet portions or panels 2b, 2c preferably are attached by, e.g., an adhesive, to the outer edges 36 of the inner top sheet portion or panel 2a. At the point of connection with the outer edges 36 of the inner top sheet portion or panel 2a, the inner regions 34 of the outer top sheet portions or panels 2b, 2c extend upwardly to form waste containment flaps 12. The waste containment flaps 12 may be formed of the same material as the outer top sheet portions or panels 2b, 2c, as in the embodiment shown. The waste containment flaps 12 may also be formed from separate elasticized strips of material that are associated with the top sheet, back sheet or both, or otherwise integrated into the garment.

The waste containment flaps 12 may be treated with a suitable surfactant to modify their hydrophobicity/hydrophilicity or imbue them with skin wellness products as desired. The central top sheet portion or panel 2a may extend past the connection point with the waste containment flaps 12 and even extend to the periphery of the back sheet. Still further, the central top sheet portion or panel 2a could extend fully between the outer top sheet portions or panels 2b, 2c and even beyond so that the outer edges 36 of the central top sheet portion or panel 2a are coextensive with and sandwiched between the outer top sheet portions or panels 2b, 2c and the back sheet 4.

Each waste containment flap 12 preferably includes a portion that folds over onto itself to form an enclosure. One or more elastic members 14 (FIG. 2) may be secured in the enclosure in a stretched condition with adhesive. As is well known in the art, when the flap elastic(s) 14 attempts to assume the relaxed, unstretched condition, the waste containment flaps 12 rise above the surface of the central top sheet portion or panel 2a.

The waist elastics 30a, 30b may be similar structures or different to impart similar or different elastic characteristics to the front and back waist portions of the diaper. In general, the waist elastics may comprise elastically extensible foam strips positioned at the front and back waist sections 22, 24. The foam strips are preferably about 0.50 inches to about 1.50 inches wide and about 3 inches to about 6 inches long. The foam strips are preferably positioned between the top sheet portions or panels and the back sheet 4. Alternatively, a plurality of elastic strands may be employed as waist elastics rather than foam strips. The foam strips are preferably polyurethane, but could be any other suitable material that preferably decreases waist band roll over, reduces leakage over the waist ends of the absorbent garment, and generally improves comfort and fit. The front and back waist foam strips 30a, 30b are stretched 50-150%, preferably 100% before being adhesively secured between the back sheet 4 and top sheet 2.

In any or all of the foregoing embodiments, the top sheet may comprise a single sheet of material having different characteristics (e.g., liquid-imperviousness/perviousness and/or hydrophobicity/hydrophilicity) and have regions of transition or demarcation therebetween.

Each leg opening 28a, 28b may be provided with a leg elastic containment system 8, sometimes referred to as conventional leg gathers. In a preferred embodiment, three strands of elastic threads are positioned to extend adjacent the leg openings 28a, 28b between the outer top sheet portions or panels 2b, 2c and the back sheet 4. The selection of appropriate elastics and the construction of leg elastic containment systems is known in the art. For example, the leg elastics 8 may be ultrasonically bonded, heat/pressure sealed using a variety of bonding patterns, or glued to the diaper 10 with adhesive.

Various commercially available materials may be used for the leg elastics 8 and elastic members 14, such as natural rubber, butyl rubber or other synthetic rubber, urethane, elastomeric materials such as spandex, which is marketed under various names, including LYCRA™ (DuPont), GLOSPAN™ (Globe) and SYSTEM 7000™ (Fulflex), and so on. The invention is not limited to any particular elastic.

The fastening system of the diaper 10 may be attached to the back waist region 24, and preferably comprises tape tabs or mechanical fasteners 32. However, any fastening system known in the art will be acceptable. Moreover, the fastening system may include a reinforcement patch below the front waist portion so that the diaper may be checked for soiling without compromising the ability to reuse the fastener. Alternatively, other diaper fastening systems are also possible, including safety pins, buttons, and snaps. Fastening systems are known in the art, and the invention is not limited to using any particular fastening system, and may be constructed without any fastening system at all, such as in training pant-type garments.

As stated previously, the invention has been described in connection with a diaper. The invention, however, is not intended to be limited to application only in diapers. Specifically, the invention may be readily adapted for use in other absorbent garments besides diapers, including, but not limited to, training pants, feminine hygiene products and adult incontinence products.

The underlying structure beneath the top sheet 2 may include, depending on the diaper construction, various combinations of elements, but in each embodiment, it is contemplated that the absorbent garment will preferably include an absorbent core 6. The absorbent core 6 may be comprised of one or more layers of material, such as an absorbent layer for storing fluids and an acquisition layer for distributing fluids. Such multiple layer absorbent cores are known in the art and disclosed in U.S. Pat. No. 5,439,458 issued to Noel et al., which is incorporated herein by reference in its entirety, and in a manner consistent with this disclosure.

The absorbent core 6 may be made from any absorbent material or materials known in the art, with the absorbent layer comprising core composite material. In one embodiment of the invention, the core composite material comprises wood fibers or other fibers such as chemical wood pulp, fibrous absorbent gelling material, or any other suitable liquid absorbing material, such as commercially available fluff pulp or fluffed bleached kraft softwood pulp or fibrous absorbent gelling material. In another embodiment of the invention, the core composite material comprises a combination of a porous fibrous web and super absorbent particles. Absorbent cores are known in the art and are disclosed, for example, in U.S. Pat. Nos. 5,281,207 and 6,068,620, issued to Chmielewski et al., U.S. Pat. No. 4,610,678 issued to Weisman et. al., U.S. Pat. No. 5,137,537 issued to Herron et. al., and U.S. Pat. No. 5,147,345 issued to Young et. al., the disclosures of which are incorporated herein by reference in their entirety in a manner consistent with this disclosure. In such an embodiment, the core composite material along with other components or layers may be encased between layers of a liquid pervious tissue over-wrap or other material to form the absorbent core 6. The absorbent core 6 may be associated with the top sheet 2, the back sheet 4, or any other suitable part of the garment 10 by any method known in the art, in order to fix the absorbent core 6 in place. Preferably, the absorbent core 6 is glued to the diaper 10 with adhesive.

The absorbent core 6 generally is elongated along the longitudinal axis 100 of the garment, and may extend along either or both of the lateral and longitudinal axes 102, 100 to the outer perimeter of the garment. In the embodiment depicted in FIG. 1, the absorbent core 6 is substantially rectangular in shape, however, it may also have rounded ends or other shapes, such as an “I” shape or a “T” shape. The absorbent core 6 may also have channels, grooves or pockets, and may have a varying thickness. The shape of the absorbent core 6 may be selected to provide the greatest absorbency in target areas where body fluids are most likely to strike the diaper 10, which is often referred to as zoned absorbency.

The absorbent core 6 typically is formed from a composite, comprising the core composite material, which may be purchased as bulk roll goods. Alternatively, the core composite material may be formed in a forming chamber from a fiberized material with SAP particles incorporated therein. In the general practice of forming fibrous materials into core composite materials, it is common to utilize a fibrous sheet of cellulosic fibers, or other suitable fibers, which is fiberized in a conventional fiberizer or other device to form discrete fibers. The discrete fibers then are entrained in an air stream or airflow along with an amount of SAP particles and directed to a forming surface where the fibers and SAP particles are deposited to form a pad of fluff, (i.e. a non-woven mat of randomly arrayed fibers containing interstitial void space and being highly compressible in character). The forming surface rotates at a speed adjusted as necessary to form the desired thickness of the pad of fluff.

Typically, the air stream with the entrained discrete fibers is directed into one end of a forming chamber, where a forming surface is located on the other end of the forming chamber. The SAP particles usually are introduced into the forming chamber downstream of the point from where the discrete fibers are introduced, yet upstream of the forming surface. The SAP particles and discrete fibers mix in the air flow in the forming chamber before they reach the forming surface.

The forming surface utilized in such systems typically is constructed as a wire or screen grid and typically employs pneumatic flow means such as a vacuum suction apparatus to define a differential pressure zone on the forming surface and impose a pressure differential thereon. The air entrained fiber and SAP particle stream typically pass through the openings or perforations in the screened grid of the forming surface. The use of vacuum suction to draw the air entrained fiber and SAP particles stream to the forming surface, with the passage of the air component through the forming surface, is highly efficient and lends itself to high speed commercial operations. A typical configuration for feeding SAP particles to an airstream containing fibers, and then to a forming rotating drum is disclosed in U.S. Pat. No. 6,139,912, the disclosure of which is incorporated by reference herein in its entirety in a manner consistent with this disclosure.

In the embodiment shown in FIG. 1, a two (2) inch diameter circle substantially having as its center the insult point 45 of the absorbent core 6 is depicted as the front pad 40. It is important to note that the location of the front pad 40 of interest varies between absorbent articles 10, according to the application of the article. Thus, since the front pad 40 may not be found in the same physical location of every absorbent core 6 and absorbent article 10, the front pad 40 should be located before sampling to fully implement the teachings of the invention. One skilled in the art can easily determine the probable insult point 45, and thus the front pad 40, of an absorbent core 6 and an absorbent article 10 without undue experimentation.

The front pad 40 encompasses a two (2) inch diameter circle having as its center the probable insult point 45 of body exudate with the absorbent article 10, and therefore the most likely entry point for a significant amount of body exudate into the absorbent core 6. Leakage protection can be optimized by designing the front pad 40 to have a total absorptive capacity of at least about 32 grams of a 1.0 weight % NaCl solution after ten (10) minutes of contact with the solution while under a restraining pressure of about 0.5 psi. A restraining pressure of about 0.3 to about 0.7 psi, and preferably about 0.5 psi, has been found by the inventors to best approximate the pressure placed on the absorbent core 6 while under usage by a wearer of an absorbent article 10, and thus the preferred restraining pressure to use while measuring absorptive capacity of the front pad 40.

Once the location of the front pad 40 has been determined, one or more samples of the absorbent article 10 are taken from the front pad 40 and measured for absorptive capacity according to the guidelines provided herein. Those skilled in the art recognize that more than one (1) sample may be taken to average a representative number of samples from the front pad 40. Protection against leakage is optimized if the front pad 40 has an average absorptive capacity of at least about 32 grams as provided. Notably, the absorptive capacity of the other portions of the absorbent core 6 do not significantly affect the urine leakage results if the front pad 40 total absorptive capacity is at least about 32 grams.

A number of techniques may be used to provide an optimized front pad 40 to improve leakage protection. For example, high AUL SAP may be disposed in the front pad 40 of the absorbent core 6 to provide the desired front pad total absorptive capacity of at least about 32 grams. Alternatively, high concentrations of lower AUL SAP may be disposed in the front pad 40 of absorbent core 6 to provide the desired front pad total absorptive capacity of at least about 32 grams. In one embodiment, the front pad 40 has a higher amount by weight of core composite material than in the other areas of the absorbent core 6 to provide the desired total absorptive capacity of at least about 32 grams. For example, the ratio of the weight of core composite material in the front pad 40 to the weight of core composite material in the remainder of the absorbent core 6 may be from about 2:1 to about 4:1.

Significantly, this technique of designing an absorbent core 6 to provide a high total absorptive capacity front pad 40 allows absorbent articles 10 with improved leakage protection to be assembled using the same amount of SAP, or even less amounts of SAP, than have been previously used in lesser-performing absorbent articles (e.g., absorbent articles having poorer leakage performance). Thus, efficient placement and use of SAP allows for a better performing article at the same or perhaps even lower cost.

As shown in FIG. 2, an additional layer 20 may be disposed between the top sheet 2 and absorbent core 6 and/or other additional layers may be disposed between these layers, or between absorbent core 6 and back sheet 4. The at least one additional layer can be any layer selected from a fluid acquisition layer, a distribution layer, an additional fibrous layer optionally containing SAP, a wicking layer, a storage layer, a surge management layer, or combinations and fragments of these layers. Such layers may be provided to assist with transferring fluids to the absorbent core 6, handling fluid surges, preventing rewet, containing absorbent material, improving core stability, or for other purposes. Skilled artisans are familiar with the various additional layers that may be included in absorbent article, and the invention is not intended to be limited to any particular type of materials used for those layers. Rather, the invention encompasses all types of wicking layers, all types of distribution layers, etc., to the extent that type of layer 20 is utilized.

The additional layers 20 may be bonded to the top sheet 2, the absorbent core 6, other additional layers 20, or any other suitable part of the garment 10 by any method known in the art, in order to fix the additional layers 20 in place. Preferably, the additional layers 20 are glued to the diaper 10 with adhesive.

One element that is useful as an additional layer 20 in the absorbent article 10 of the invention is a fluid acquisition layer, or fluid handling layer. The fluid acquisition layer 20 typically comprises a hydrophilic fibrous material, and serves to quickly collect and temporarily hold discharged body fluid. A portion of discharged fluid may, depending upon the wearer's position, permeate the acquisition layer 20 and be absorbed by the absorbent core 6 in the area proximate to the discharge. However, since fluid is frequently discharged in gushes, the absorbent core 6 in such area may not absorb the fluid as quickly as it is discharged. Therefore, the fluid acquisition layer 20 hereof also facilitates transport of the fluid from the point of initial fluid contact to other parts of the absorbent core 6. In the context of the invention, it should be noted that the term “fluid” includes, but is not limited to, liquids, urine, menses, perspiration, and water-based body fluids.

The function of the fluid acquisition layer 20 is relatively important. The fluid acquisition layer 20 preferably has sufficient capillary suction to more fully drain the top sheet 2 and yet not exhibit excessive fluid retention to make it difficult for the fibrous structure to desorb the acquisition layer 20. The acquisition layer 20 may be comprised of any or a combination of several different materials including nonwoven or woven webs of synthetic fibers including polyester, polypropylene, or polyethylene, natural fibers including cotton or cellulose, blends of such fibers, foams, fluff pulp, apertured films, or any equivalent materials or combinations of materials.

Another useful layer 20 for use in the absorbent garment 10 of the invention includes a fluid distribution layer 20. Fluid distribution layer 20 of the invention can include any combination or all of three basic components: chemically stiffened, twisted, and curled bulking fibers, high surface area fibers, and binder fibers. In a preferred embodiment of the invention, fluid distribution layer 20 comprises from about 20% to about 80% of the chemically stiffened, twisted, and cured fibers, from about 10% to about 80% of a high surface area fiber, and from 0% to about 50% of a thermoplastic binding means for increasing physical integrity of the web. All percentages herein refer to weight percentages based on total dry web weight. Preferably, the fluid distribution layer 20 will comprise between about 45% and about 60% of chemically stiffened, twisted, and cured fibers, between about 5% and about 15% of a hot melt fibrous binding means, and between about 30% and about 45% high surface area cellulose binding means. More preferably, the fluid distribution layer 20 comprises about 10% thermoplastic binding means, about 45% chemically stiffened, twisted, and cured fibers, and about 45% high surface area fibers.

Chemical additives also can be used as binding means, and are incorporated into the acquisition/distribution layer at levels typically of about 0.2% to about 2.0%, dry web weight basis. The three basic fiber components are described in greater detail in U.S. Pat. No. 5,549,589, the disclosure of which is incorporated by reference herein in its entirety, and in a manner consistent with this disclosure.

Fluid distribution layer 20 also may be comprised of non-woven or woven webs of synthetic fibers, natural fibers, foams, carded, thermal bonded materials, and the like.

Another useful layer in the absorbent article 10 of the invention includes a storage layer 20. Such storage layers 20 typically have limited transport and wicking capabilities but high storage or retention capacity, and rely upon the fibrous structure of the absorbent core 6 to distribute incoming fluid over a larger area. It is preferred to dispose storage layer 20 between the absorbent core 6 and the back sheet 4, or between the first and second tissue layers 16, 18 that optionally surround absorbent core 6.

Storage layers or members 20 may be of generally conventional design and composition, selected with regard to the particular application. The storage layer or member 20 may be monolayer or multilayer, homogeneous or stratified, profiled or uniform, etc. Materials suitable for use in such storage layers 20 may be natural or synthetic in origin, woven, non-woven, fibrous, cellular, or particulate, and may include particles, layers, or regions of absorbent polymeric gelling materials. Other preferred materials include fluff pulp and SAP composites, either air laid or wet laid, and high capacity resilient foam materials. Storage layer 20 may also have any desired size and/or shape as may prove suitable for a particular application, including square, rectangular, oval, elliptical, oblong, etc. They may also take on a three-dimensional shape or may be substantially planar in nature.

Another useful layer 20 in absorbent article 10 is a wicking layer 20. Wicking layers usually have both fluid acquisition and fluid distribution properties. For example, vertical wicking, which is in general the ability to transport fluids vertically from the top sheet 2 to the absorbent core 6, is related in many respects to fluid acquisition. Horizontal wicking, which is in general the ability to transport fluids along the longitudinal 100 and lateral 102 axes of FIG. 1, is related in many respects to fluid distribution.

Any conventional wicking materials can be used for the wicking layer 20 of the invention. Typical suitable wicking layer 20 materials include, for example, high-density air laid fluff pulps, high-density wet laid fluff pulp, multi-groove fibers, and the like. In addition, high internal phase emulsion (HIPE) foams such as those disclosed in U.S. Pat. No. 5,650,222 can be used, braided materials such as those disclosed in H1,585, and other conventional fibrous and strand materials can be used. The disclosures of U.S. Pat. No. 5,650,222 and H1,585 are incorporated by reference here in their entirety, and in a manner consistent with the invention. Wicking layer 20 also may be comprised of two or more sublayers containing absorbent materials with differing wicking characteristics. Those skilled in the art will be able to include a suitable wicking layer 20 in an absorbent garment manufactured according to the invention without undue experimentation based on the teachings herein.

Various combinations of any of the above-mentioned layers also may be used as the additional layer 20. For example, additional layer 20 may comprise a combination of a wicking layer and a distribution layer, thereby imparting the additional layer 20 with both wicking and distribution properties. Skilled artisans will be capable of designing additional layers 20 to have desired properties by combining various layer attributes, or by fragmenting the layer.

The dimensions of additional layer(s) 20 may be the same as or different from the dimensions of the absorbent core 6 and/or upper layer 20. It is preferred that additional layer(s) 20 have a width in the lateral direction (102) of anywhere from about 10 mm to about 100 mm, and preferably from about 25 mm to about 80 mm.

The test methods for determining the location of the insult point for an absorbent article and measuring front pad absorptive capacity are set forth below.

Testing Methods

Estimation of Insult Point of Absorbent Article

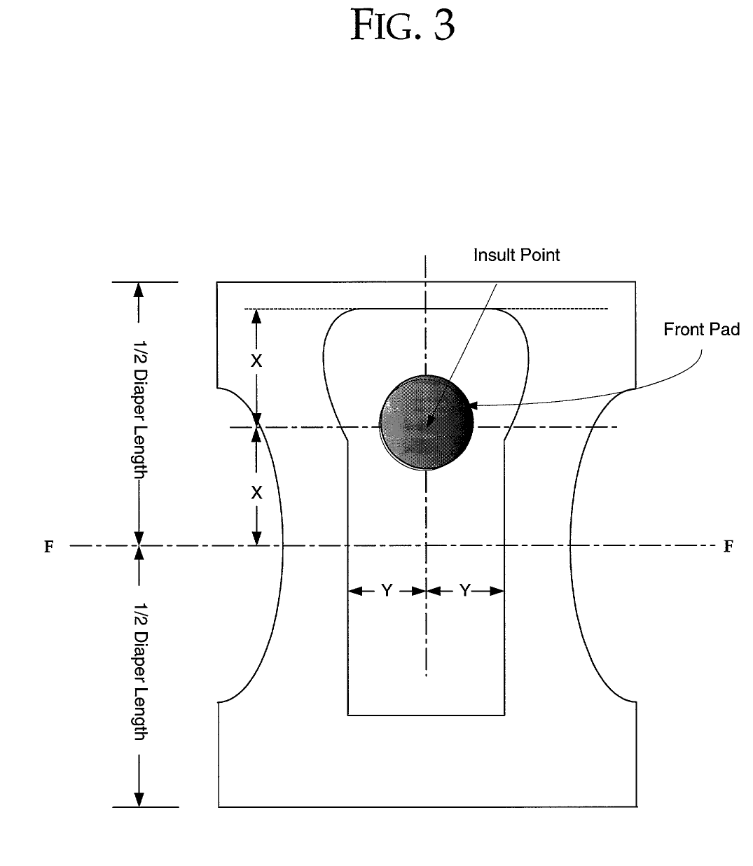

Referring now to FIG. 3, one method of determining the insult point is presented. An absorbent article to be tested is laid flat. First, the ½-way longitudinal length in the cross-direction is determined, depicted as typically being located substantially along fold line F-F in FIG. 3. This is also the half way point in terms of diaper length. The distance from that line to the front edge of the absorbent core then is determined, and the half-way point of that distance, designated by the letter “X” in FIG. 3, is the longitudinal coordinate for the insult point. The lateral coordinate for the insult point can be determined as the center of the lateral width of the absorbent core (the lateral width of the core in FIG. 3 is divided in halves as denoted by the letters “Y”).

Samples for measurement of total absorptive capacity are taken from the two (2) inch diameter circle having substantially as its center the insult point. For the test method described below, one circular sample having a diameter of 2 inches (5.08 cm) is taken substantially from the insult point as seen in FIG. 3. More samples may be taken, and may be obtained in any number of ways. For example, in one manner, the edges of the additional circular samples may be substantially adjacent the edge of the cut-out of the first sample that is substantially centered around the insult point. Other methods will be readily apparent to those skilled in the art.

Test Method for Front Pad Total Absorptive Capacity Measurement

The Front Pad Total Absorptive Capacity Test Method is used to measure the amount of a 1.0 weight % NaCl solution absorbed, on average, in ten (10) minutes by the front pad of a sample of an absorbent article while the sample is subjected to a restraining pressure of 0.5 psi. The absorptive capacity is measured as gm of solution absorbed during ten (10) minutes of exposure to the solution under testing conditions.

A 1.0 weight % NaCl solution is prepared by weighing approximately 50 gm NaCl into a weighing dish, and then transferring the 50 gm NaCl to a 5000 ml container. The 5000 ml container is filled with deionized water and mixed to prepare a 1.0 weight % saline solution.

An absorbent article is selected as a sample to be tested. A circular sample approximately two (2) inches in diameter is removed from substantially in or around the insult point of the absorbent article. The sample is cut from the absorbent article from the insult point using a two (2) inch diameter stainless steel metal circular die that is approximately 1⅝ inches high. Any cutting patterns may be used as well as long as the average total absorptive capacity of the front pad of the sample is measured.

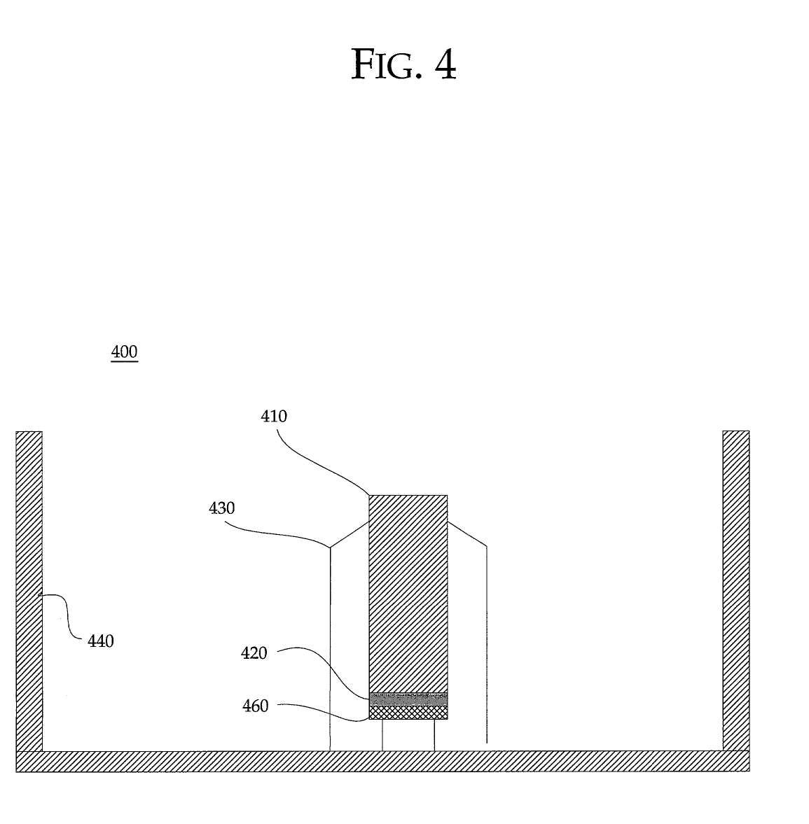

The test utilizes an apparatus 400 as seen in FIG. 4. The sample holder 430 is a cylindrical nylon basket, approximately two (2) inches in depth and approximately three (3) inches in diameter, with a cylindrical hole bored in the center of the sample holder that slightly exceeds two (2) inches in diameter in order to accommodate the two (2) inch diameter sample 420. The sample holder 430 has four (4) ½ inch stainless steel feet and an eight (8) inch long wire handle attached to the sample holder on substantially opposite sides with stainless steel eyes. A 16 gauge stainless steel perforated plate 460 with ⅛ inch holes, and having an open area of approximately 40%, is placed at the bottom of the sample holder 430.

Each sample is weighed, then placed into a sample holder with the top sheet facing downward toward the metal perforated plate 460. The weight of the filled sample holders 430 is measured. A load of approximately 0.5 psi is applied on top of each sample 420 in each filled sample holder 430 by placing a substantially two (2) inch in diameter cylindrical stainless steel weight 410 on top of the sample. For a 0.5 psi load, the weight 410 is approximately 1¾ inches high. The weight 410 is machined to a size such that it closely approximates the cross sectional area of the sample 420 and yet may freely move up or down in the sample holder 430 during the test.

A sample box 440 is prepared for holding the filled sample holders 430. The sample box 440 is approximately ¼ inch thick plexiglass that is approximately 20 inches in width, 14½ inches in length, and 5 inches deep, and is water tight. Saline solution to cover the filled sample holders 430 is added to the sample box 440, which is approximately 120 ml more than the volume of saline solution required to rise ⅛ inch above the tops of the empty sample holders 430 when the empty sample holders 430 are placed into the sample box 440. The temperature should be maintained at about 75° F. plus or minus about 2 degrees.

The filled sample holders 430 are placed in the filled sample box 440 at substantially the same time. After ten (10) minutes, the filled sample holders 430 are removed from the sample box 440 and allowed to drip for one (1) minute. The weight 410 is removed from the filled sample holders 430.

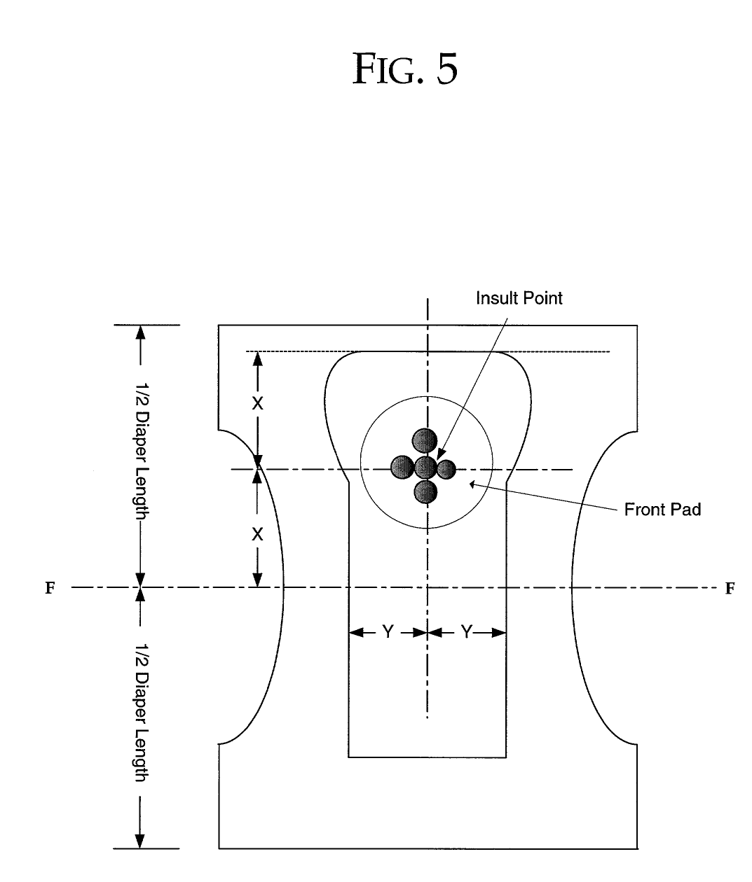

The filled sample holders are weighed. The total absorptive capacity of each sample, in gm of solution absorbed, is the weight of solution absorbed by the sample during the ten (10) minute exposure to the 1.0 weight % saline solution while under a restraining pressure of 0.5 psi. The front pad total absorptive capacity is calculated by determining the difference in the weight of the filled sample holder measured after and before the test. If multiple samples are taken from one absorbent article, the average total absorptive capacity of the front pad is determined by averaging front pad total absorptive capacities. FIG. 5 illustrates an alternative pattern for cutting multiple samples from the absorbent article to measure average total absorptive capacity, although any pattern may be used. As conducted in the following Examples, one sample was taken and reported as the total absorptive capacity.

The invention is further illustrated by the following Examples which should not be regarded as limiting.

### EXAMPLE 1

The insult points of several absorbent cores were determined according to the test method previously described. Samples of core materials were taken from the front pad of each of numerous absorbent articles, both experimental and commercial, and tested to determine their respective total absorptive capacities, using the method previously set forth. The total absorptive capacities for the samples are summarized in Table 1.

TABLE 1
Absorptive Capacity in Front Pad, grams of solution absorbed,
10 minutes exposure to a 1.0 weight % NaCl solution under
0.5 psi restraining pressure

| | | TOTAL ABSORPTIVE |
|---|---|---|
| SAMPLE | SAMPLE SOURCE | CAPACITY, g |
| A | Experimental | 23.5 |
| B | Experimental | 23.4 |
| C | Experimental | 23.0 |
| D | Experimental | 24.6 |
| E | Experimental | 25.4 |
| F | Experimental | 28.8 |
| G | Experimental | 33.0 |
| H | Experimental | 23.2 |
| I | Experimental | 25.0 |
| J | Experimental | 24.6 |
| K | Experimental | 27.6 |
| L | Experimental | 30.4 |
| M | Experimental | 32.0 |
| N | Commercial | 31.4 |
| O | Commercial | 29.8 |
| P | Commercial | 22.0 |
| Q | Commercial | 25.8 |
| R | Commercial | 26.1 |
| S | Commercial | 24.3 |
| T | Control | 25.0 |

The samples exhibited total absorptive capacities ranging from about 22 grams solution for Commercial Sample P to about 33 grams solution for Experimental Sample G. Two experimental samples, G and M, exhibit front pad total absorptive capacities of at least 32 grams solution, while no commercial samples had such front pad total absorptive capacities.

### EXAMPLE 2

Four Experimental Samples (J, K, L, and M) were measured for urine leakage under actual use conditions. This testing involves home use of samples by panels of 50 babies, where each panel includes ½ female babies and ½ male babies. The caregivers of the babies report, for those tested samples containing urine exudate only, whether the samples leak or not, and the type of use (day, day napping, night) for the sample.

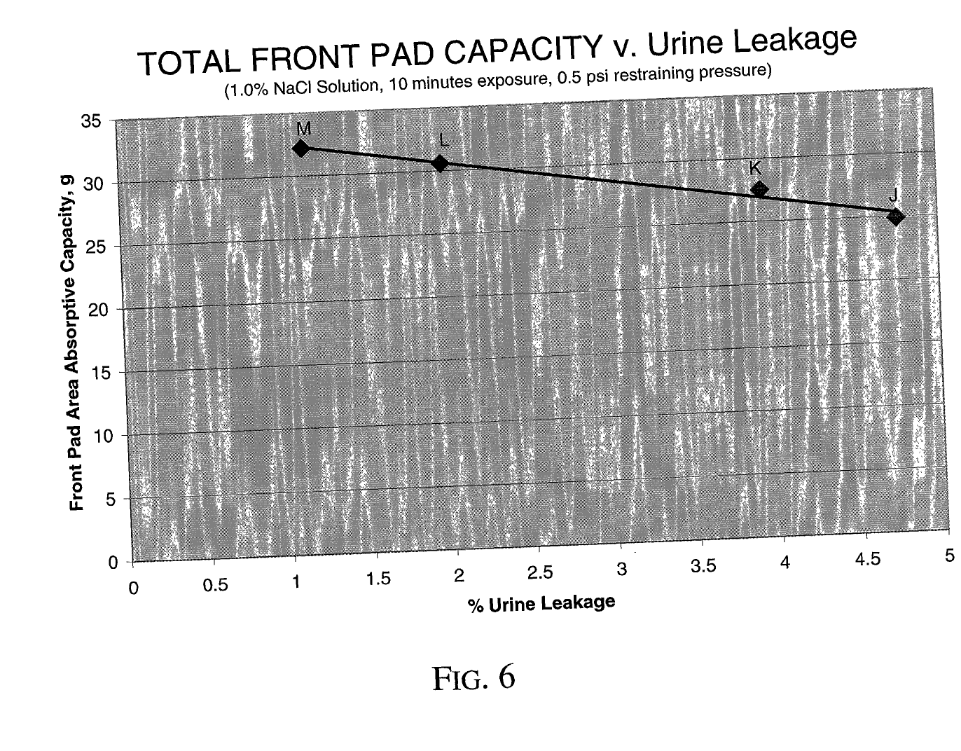

The front pad total absorptive capacities of Samples J, K, L, and M were correlated to the percentage on average of the samples in the panels that experienced urine leakage, as shown in FIG. 6. Correlation is close between front pad total absorptive capacity and percentage of samples that experience urine leakage.

It is to be understood that the embodiments and variations shown and described herein are merely illustrative of the principles of this invention and that various modifications may be implemented by those skilled in the art without departing from the scope and spirit of the invention.

What is claimed is:

1. An absorbent core comprising:
a composite; and
a wrapping material substantially adjacent to at least some of the outer surface of the composite,
the absorbent core being associated with an absorbent article, wherein the absorbent core further comprises a front pad and a back pad, the front pad having an absorptive capacity of at least about 32 grams of an aqueous solution containing 1.0 weight % sodium chloride absorbed after ten minutes of contact with the aqueous solution while under a restraining pressure of about 0.5 psi.

2. The absorbent core of claim 1, wherein the front pad comprises a two inch diameter circle having substantially as its center the insult point of the absorbent core when the absorbent core is used in an absorbent article for the containment of body exudate discharged by the body of a wearer of the absorbent article.

3. The absorbent core of claim 2, wherein the front pad has an absorptive capacity of at least about 35 grams.

4. The absorbent core of claim 2, wherein the front pad has an absorptive capacity of at least about 40 grams.

5. The absorbent core of claim 2, wherein the front pad has an absorptive capacity of at least about 50 grams.

6. The absorbent core of claim 2, wherein the front pad has an absorptive capacity of at least about 60 grams.

7. The absorbent core of claim 2, wherein the front pad has an absorptive capacity between about 32 to about 50 grams.

8. The absorbent core of claim 1, wherein the absorptive capacity of the front pad of the absorbent article differs from and is greater than the absorptive capacity of the back pad.

9. The absorbent core of claim 1, wherein the composite comprises a porous fiber matrix and a superabsorbent material.

10. The absorbent core of claim 1, wherein the front pad comprises an amount of superabsorbent material greater than the amount of superabsorbent material in the back pad.

11. The absorbent core of claim 1, wherein the porous fiber matrix comprises wood pulp fluff.

12. The absorbent core of claim 1, wherein the superabsorbent material comprises natural or synthetic polymers with hydrophilic groups, sodium neutralized cross-linked polyacrylates and polysaccharides, carboxylated, phosphonoalkylated, sulphoxylated or phosphorylated cellulose, starch, and regenerated cellulose, water soluble acrylic or vinyl monomers crosslinked with a polyfunctional reactant, starch modified polyacrylic acids, hydrolyzed polyacrylonitrile and their alkali metal salts, and mixtures thereof.

13. The absorbent core of claim 1, wherein the absorbent core comprises superabsorbent material in an amount of from about 35% to about 95% weight percent based on the total weight of the absorbent core.

14. The absorbent core of claim 1, wherein the ratio of the weight of composite in the front pad to the weight of composite in the back pad is about 2 to about 4.

15. An absorbent article, comprising:
a top sheet material;
a back sheet material; and
an absorbent core disposed between the top sheet material and the back sheet material;
wherein the absorbent core comprises:
a composite; and
a wrapping material substantially adjacent to at least some of the outer surface of the composite,
wherein the absorbent core further comprises a front pad and a back pad, the front pad having an absorptive capacity of at least about 32 grams of an aqueous solution containing 1.0 weight % sodium chloride absorbed after ten minutes of contact with the aqueous solution while under a restraining pressure of about 0.5 psi.

16. The absorbent article of claim 15, wherein the front pad comprises a two inch diameter circle having substantially as its center the insult point of the absorbent core when the absorbent core is used in an absorbent article for the containment of body exudate discharged by the body of a wearer of the absorbent article.

17. The absorbent article of claim 16, wherein the front pad has an absorptive capacity of at least about 35 grams.

18. The absorbent article of claim 16, wherein the front pad has an absorptive capacity of at least about 40 grams.

19. The absorbent article of claim 16, wherein the front pad has an absorptive capacity of at least about 50 grams.

20. The absorbent article of claim 16, wherein the front pad has an absorptive capacity of at least about 60 grams.

21. The absorbent core of claim 16, wherein the front pad has an absorptive capacity between about 32 to about 50 grams.

22. A method of making an absorbent article, the method comprising:
preparing a top sheet material;
preparing a back sheet material;
preparing an absorbent core; and
disposing the absorbent core at least partially between the top sheet material and the back sheet;
wherein the absorbent core comprises a front pad and a back pad, the front pad having an absorptive capacity of at least about 32 grams, the absorptive capacity being the amount of an aqueous solution containing 1.0 weight % sodium chloride absorbed after ten minutes of contact with the aqueous solution while under a restraining pressure of about 0.5 psi.

23. The method of claim 22, wherein the front pad comprises a two inch diameter circle having substantially as its center the insult point of the absorbent core when the absorbent core is used in an absorbent article for the containment of body exudate discharged by the body of a wearer of the absorbent article.

24. A method of making an absorbent article, the method comprising:
preparing a chassis;
preparing an absorbent core; and
operatively associating the chassis and the core,
wherein the absorbent core comprises a front pad and a back pad, the front pad having an absorptive capacity of at least about 32 grams of an aqueous solution containing 1.0 weight % sodium chloride absorbed after ten minutes of contact with the aqueous solution while under a restraining pressure of about 0.5 psi.

25. The method of claim 24, wherein the front pad comprises a two inch diameter circle having substantially as its center the insult point of the absorbent core when the absorbent core is used in an absorbent article for the containment of body exudate discharged by the body of a wearer of the absorbent article.

26. A method of providing leakage protection in an absorbent article, the method comprising:
preparing a top sheet material;
preparing a back sheet material;
preparing an absorbent core; and
disposing the absorbent core at least partially between the top sheet material and the back sheet material,
wherein the absorbent core comprises a front pad and a back pad, the front pad having an absorptive capacity of at least about 32 grams of an aqueous solution containing 1.0 weight % sodium chloride absorbed after ten minutes of contact with the aqueous solution while under a restraining pressure of about 0.5 psi.

27. The method of claim 26, wherein the front pad comprises a two inch diameter circle having substantially as its center the insult point of the absorbent core when the absorbent core is used in an absorbent article for the containment of body exudate discharged by the body of a wearer of the absorbent article.

28. A method of designing an absorbent core to provide good leakage protection, the method comprising:
preparing an absorbent core, the core comprising a composite;
determining the insult point of the absorbent core, the insult point located proximately in the area of contact of body exudate with the absorbent core when a substantially similar absorbent core is used in an absorbent article for the containment of body exudate discharged by a wearer of the absorbent article; and
varying the weight and/or composition of the composite in a front pad of the absorbent core, the front pad comprising a two inch diameter circle having substantially as its center the insult point, until the absorptive capacity of the front pad is on average at least about 32 grams of an aqueous solution containing 1.0 weight % sodium chloride absorbed after ten minutes of contact with the aqueous solution while under a restraining pressure of about 0.5 psi.

29. A method of designing an absorbent core that provides good leakage protection, the method comprising:
preparing an absorbent core, the absorbent core comprising a composite;
determining the insult point of the absorbent core, the insult point located proximately in the area of contact of body exudate with the absorbent core when a substantially similar absorbent core is used in an absorbent article for the containment of body exudate discharged by a wearer of the absorbent article;
varying the weight and/or composition of the composite in a front pad of the absorbent core, the front pad comprising a two inch diameter circle having substantially as its center the insult point, until the absorptive capacity of the front pad on average is at least about 32 grams of an aqueous solution containing 1.0 weight % sodium chloride absorbed after ten minutes of contact with the aqueous solution while under a restraining pressure of about 0.5 psi; and
determining the lowest cost absorbent core with a front pad absorptive capacity of at least about 32 grams.
